(19) United States
(12) Patent Application Publication
(10) Pub. No.: US 2024/0279081 A1
(43) Pub. Date: Aug. 22, 2024

(54) SPACE CLEANING DEVICE

(71) Applicant: Panasonic Intellectual Property Management Co., Ltd., Kadoma-shi, Osaka (JP)

(72) Inventors: Takahisa AKAGAWA, Aichi (JP); Jumpei KOSEDO, Aichi (JP)

(21) Appl. No.: 18/570,752

(22) PCT Filed: May 16, 2022
(86) PCT No.: PCT/JP2022/020376

§ 371 Date: Dec. 15, 2023

(30) Foreign Application Priority Data
Jun. 21, 2021 (JP) 2021-102122

(51) Int. Cl. C02F 1/461; A61L 9/14; C02F 1/467; F24F 8/192

(52) U.S. Cl. CPC C02F 1/4618; A61L 9/145; C02F 1/4674; F24F 8/192; A61L 2209/111; A61L 2209/213; C02F 2001/46185; C02F 2201/4614; C02F 2201/4619

(57) ABSTRACT

Space cleaning device (1000) of the present disclosure includes: an electrolysis tank; electrolysis accelerator inputter (400) that inputs an electrolysis accelerator; electrolyzed water generator (460) that generates the electrolyzed water; a cleaner; input instruction unit (510) that instructs input of the electrolysis accelerator; input detector (512) that detects the input of the electrolysis accelerator; non-input detection number counter (514); and electric conduction setting determiner (522). Non-input detection number counter (514) counts the non-input detection number that is the number of times input detector (512) has not detected the input of the electrolysis accelerator even after input instruction unit (510) has instructed the input of the electrolysis accelerator. Electric conduction setting determiner (522) determines electric conduction setting for electrolysis in the electrolyzed water generator based on the non-input detection number counted by non-input detection number counter (514).

### TECHNICAL FIELD

The present disclosure relates to a space cleaning device.

### BACKGROUND ART

In order to remove (including inactivate) bacteria, fungi, viruses, odors, and the like in the air, a space cleaning device that generates and releases electrolyzed water containing hypochlorous acid by electrolysis is known (see, for example, PTL 1). For generation of hypochlorous acid, it is necessary to input an electrolysis accelerator such as a salt to water that is a target of electrolysis to generate water containing chloride ions.

### CITATION LIST

### Patent Literature

PTL 1: Unexamined Japanese Patent Publication No. 2019-24811

### SUMMARY OF THE INVENTION

When the electrolysis accelerator is automatically input in the space cleaning device, a function of detecting input of the electrolysis accelerator is required. For example, a light emitter and a light receiver that receives light from the light emitter are disposed across an input path of the electrolysis accelerator, and when the level of light reception in the light receiver decreases, input of the electrolysis accelerator is detected. In such configuration, when input is not detected even if the input of the electrolysis accelerator is instructed, the input of the electrolysis accelerator is instructed again. However, when detection of input fails even if the electrolysis accelerator is actually input, the electrolysis accelerator is additionally input. As a result, the concentration of hypochlorous acid in the electrolyzed water may become higher than a reference value.

An object of the present disclosure is to provide a technique for suppressing a concentration of hypochlorous acid in electrolyzed water from becoming higher than a reference value.

A space cleaning device of an aspect of the present disclosure includes: an electrolysis tank that stores water and electrolyzed water; an electrolysis accelerator inputter that inputs an electrolysis accelerator into the electrolysis tank; an electrolyzed water generator that generates the electrolyzed water by electrolyzing the water into which the electrolysis accelerator is input; a cleaner that brings the electrolyzed water generated by the electrolyzed water generator into contact with air sucked from an intake port; an input instruction unit that instructs the electrolysis accelerator inputter to input the electrolysis accelerator; an input detector that detects input of the electrolysis accelerator by the electrolysis accelerator inputter; a non-input detection number counter that counts a non-input detection number that is a number of times the input detector has not detected input of the electrolysis accelerator even after the input instruction unit has instructed the input of the electrolysis accelerator; and an electric conduction setting determiner that determines electric conduction setting for electrolysis in the electrolyzed water generator based on the non-input detection number counted by the non-input detection number counter.

Any combinations of the above-described components and modifications of the expressions of the present disclosure among methods, devices, systems, recording media, and computer programs are also effective as aspects of the present disclosure.

According to the present disclosure, it is possible to suppress the concentration of hypochlorous acid in electrolyzed water from becoming higher than a reference value.

### BRIEF DESCRIPTION OF THE DRAWINGS

FIG. 1 is a view illustrating an internal configuration of a space cleaning device according to an exemplary embodiment of the present disclosure.

FIG. 2A is a view illustrating an operation outline of the space cleaning device according to the exemplary embodiment of the present disclosure.

FIG. 2B is a view illustrating an operation outline of the space cleaning device according to the exemplary embodiment of the present disclosure.

FIG. 2C is a view illustrating an operation outline of the space cleaning device according to the exemplary embodiment of the present disclosure.

FIG. 3A is a view illustrating an operation outline of the space cleaning device according to the exemplary embodiment of the present disclosure.

FIG. 3B is a view illustrating an operation outline of the space cleaning device according to the exemplary embodiment of the present disclosure.

FIG. 4A is a view illustrating an operation outline of the space cleaning device according to the exemplary embodiment of the present disclosure.

FIG. 4B is a view illustrating an operation outline of the space cleaning device according to the exemplary embodiment of the present disclosure.

FIG. 5 is an exploded perspective view of an electrolysis accelerator inputter according to the exemplary embodiment of the present disclosure.

FIG. 6 is a perspective view illustrating an inside of a case of the electrolysis accelerator inputter according to the exemplary embodiment of the present disclosure.

FIG. 7 is a view illustrating functional blocks of the space cleaning device according to the exemplary embodiment of the present disclosure.

FIG. 8 is a view illustrating an outline of process in an electric conduction setting determiner according to the exemplary embodiment of the present disclosure.

FIG. 9 is a flowchart showing a control procedure by the space cleaning device according to the exemplary embodiment of the present disclosure.

FIG. 10 is a flowchart showing an electric conduction procedure by the space cleaning device according to the exemplary embodiment of the present disclosure.

### DESCRIPTION OF EMBODIMENT

Prior to specifically describing an exemplary embodiment of the present disclosure, an outline of the exemplary embodiment will be described. The present exemplary embodiment relates to a space cleaning device that generates electrolyzed water based on water and an electrolysis accelerator and then releases the electrolyzed water. When input of the electrolysis accelerator is not detected even if the input of the electrolysis accelerator is instructed in the space cleaning device, the input of the electrolysis accelerator is instructed again. For example, when detection of input fails even if the electrolysis accelerator is actually input, the electrolysis accelerator is additionally input. As a result, although only one tablet of the electrolysis accelerator should be input into water, two or more tablets of the electrolysis accelerator are input into water. In this situation, when the water to which the electrolysis accelerator is input is electrolyzed, electrolyzed water having the concentration of hypochlorous acid higher than a reference value is generated. Since the release of the electrolyzed water having the concentration of hypochlorous acid higher than a reference value is not preferable, it is required to suppress the concentration of hypochlorous acid in electrolyzed water from becoming higher than a reference value even when input of the electrolysis accelerator is instructed again.

The space cleaning device according to the present exemplary embodiment counts the number of times (hereinafter, “non-input detection number”) when input of the electrolysis accelerator is not detected even if the input is instructed, although the possibility is low. The space cleaning device adjusts the electric conduction time for electrolysis according to the non-input detection number when input is detected. For example, the more the non-input detection number increases, the more the electric conduction time is shortened. As described above, since the electric conduction time is shortened even if a large amount of the electrolysis accelerator is input, an excessive rise in the concentration of hypochlorous acid in the electrolyzed water is suppressed.

A conventional space cleaning device generates water containing chloride ions by dissolving an electrolysis accelerator into water in a water storage, and then generates electrolyzed water containing active oxygen species by electrolyzing the water containing chloride ions by electric conduction to electrodes. In the conventional space cleaning device, generated electrolyzed water and air sucked from the outside are continuously brought into contact with each other in a water storage, and then the air brought into contact is released to the outside by rotation of a fan. Therefore, electrolyzed water in the water storage is easily contaminated by contact with air. When the electrolyzed water is contaminated, the electrodes are likely to be deteriorated.

In order to suppress deterioration of the electrodes, in the space cleaning device according to the present exemplary embodiment, the water storage is divided into two water tanks of an electrolysis tank and a humidification tank. The electrolysis tank includes electrodes, and the electrodes electrolyze water containing chloride ions to generate electrolyzed water in the electrolysis tank. The electrolyzed water generated in the electrolysis tank is supplied to the humidification tank. Furthermore, in the humidification tank, the electrolyzed water from the electrolysis tank and the air sucked from the outside are continuously brought into contact with each other, and then the air brought into contact is released to the outside by rotation of a fan. With such configuration, the electrolyzed water in the electrolysis tank does not come into contact with air, making the electrolyzed water less likely to be contaminated, and the electrodes are suppressed from deteriorating.

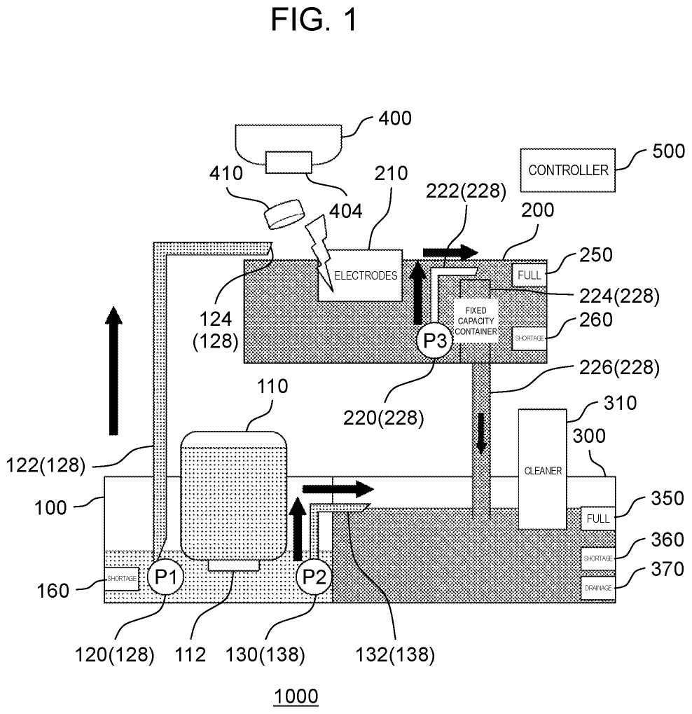

An exemplary embodiment of the present disclosure will now be explained with reference to the accompanying drawings. FIG. 1 illustrates an internal configuration of space cleaning device 1000.

Space cleaning device 1000 includes water storage tank 100, water supply tank 110, lid 112, first pump 120, first water supply pipe 122, supply port 124, second pump 130, second water supply pipe 132, water shortage float 160, electrolysis tank 200, electrodes 210, third pump 220, third water supply pipe 222, fixed capacity container 224, third water supply pipe 226, full water float 250, water shortage float 260, humidification tank 300, cleaner 310, full water float 350, water shortage float 360, drainage float 370, electrolysis accelerator inputter 400, input port 404, electrolysis accelerator 410, and controller 500. Here, first pump 120, first water supply pipe 122, and supply port 124 are included in first supplier 128, and second pump 130 and second water supply pipe 132 are included in second supplier 138. Third pump 220, third water supply pipe 222, fixed capacity container 224, and third water supply pipe 226 are included in third supplier 228. Note that controlling the operation of each component included in space cleaning device 1000 by controller 500 is also described as that controller 500 controls the operation of space cleaning device 1000. Hereinafter, (1) basic configuration, (2) initial process, (3) normal process, (4) reprocessing, and (5) input process of electrolysis accelerator will be described in this order.

### (1) Basic Configuration

Water storage tank 100 has a box shape with a top surface opened, has a structure that can store water, and stores water supplied from water supply tank 110 described later. Water storage tank 100 is disposed, for example, in a lower part of space cleaning device 1000. Water supply tank 110 is a tank that internally stores water, and is detachable from water storage tank 100. An opening (not illustrated) of water supply tank 110 is provided with lid 112, and a center of lid 112 is provided with an opening and closing portion (not illustrated). When the opening and closing portion is opened, water in water supply tank 110 is supplied to water storage tank 100.

Specifically, when water supply tank 110 is attached to water storage tank 100 with the opening of water supply tank 110 facing downward, the opening and closing portion is opened. That is, when water supply tank 110 containing water is attached to water storage tank 100, the opening and closing portion is opened to supply water to water storage tank 100, and water is stored in water storage tank 100. When the water level in water storage tank 100 rises and reaches lid 112, the water supply is stopped because the opening of water supply tank 110 is water-sealed. When water remains inside water supply tank 110, water in water supply tank 110 is supplied to water storage tank 100 every time the water level in water storage tank 100 drops. As a result, the water level in water storage tank 100 is kept constant.

First pump 120 is disposed in water storage tank 100, and is connected to first water supply pipe 122. When operating in response to an instruction from controller 500, first pump 120 pumps up water stored in water storage tank 100 toward first water supply pipe 122. First water supply pipe 122 is a pipe connecting water storage tank 100 and electrolysis tank 200, and has a supply port 124 at an end on electrolysis tank 200 side. Water pumped up by first pump 120 flows in first water supply pipe 122, and is supplied from supply port 124 to electrolysis tank 200. That is, first pump 120, first water supply pipe 122, and supply port 124 supply water from water storage tank 100 to electrolysis tank 200.

Second pump 130 is disposed in water storage tank 100, and is connected to second water supply pipe 132. When operating in response to an instruction from controller 500, second pump 130 pumps up water stored in water storage tank 100 toward second water supply pipe 132. Second water supply pipe 132 is a pipe connecting water storage tank 100 and humidification tank 300. Water pumped up by second pump 130 flows in second water supply pipe 132, and is supplied to humidification tank 300. That is, second pump 130 and second water supply pipe 132 supply water from water storage tank 100 to humidification tank 300.

Electrolysis tank 200 has a box shape with a top surface opened, and is disposed at the lower side of supply port 124. Electrolysis tank 200 stores water supplied from supply port 124. On the upper side of electrolysis tank 200, electrolysis accelerator inputter 400 is disposed side by side with supply port 124. Electrolysis accelerator inputter 400 can be loaded internally with electrolysis accelerator 410, and rotates a tablet input member (not illustrated) upon receiving an input instruction of electrolysis accelerator 410 from controller 500. When the tablet input member rotates, electrolysis accelerator 410 drops into electrolysis tank 200. Electrolysis accelerator inputter 400 counts the number of electrolysis accelerators 410 dropped into electrolysis tank 200, and stops the rotation of the tablet input member upon determining that one tablet of electrolysis accelerator 410 has dropped into electrolysis tank 200. That is, electrolysis accelerator inputter 400 inputs electrolysis accelerator 410 into electrolysis tank 200. When electrolysis accelerator 410 dissolves into water in electrolysis tank 200, water containing chloride ions is generated in electrolysis tank 200. One example of electrolysis accelerator 410 is sodium chloride and is formed as an electrolysis acceleration tablet.

Electrodes 210 is installed in a manner to be immersed in water in electrolysis tank 200. By being electrically conducted, electrodes 210 electrochemically electrolyzes water containing chloride ions in electrolysis tank 200 to generate electrolyzed water containing active oxygen species. Here, the active oxygen species means oxygen molecules having an oxidation activity higher than the oxidation activity of normal oxygen and substances related with them. For example, the active oxygen species include what is called a narrow sense of active oxygen such as superoxide anion, singlet oxygen, hydroxyl radical, or hydrogen peroxide, and what is called a broad sense of active oxygen such as ozone or hypochlorous acid (hypohalous acid).

Electrodes 210 may generate electrolyzed water by repeating one cycle a plurality of times where one cycle has electric conduction time for performing electric conduction for electrolysis and time after stopping the electric conduction, that is, non-electric conduction time that is the time when the electric conduction is not performed. By providing the non-electric conduction time for electrodes 210, the life of electrodes 210 is extended. When the electric conduction time is lengthened with respect to the non-electric conduction time, electrolyzed water containing a larger amount of active oxygen species per cycle is generated. When the non-electric conduction time is lengthened with respect to the electric conduction time, generation of active oxygen species per cycle can be suppressed. Furthermore, when the power amount in the electric conduction time is increased, electrolyzed water containing a larger amount of active oxygen species is generated. As described above, it can be said that electrolysis tank 200 is a tank for generating electrolyzed water from the water into which electrolysis accelerator 410 is input.

Third pump 220 is disposed in electrolysis tank 200, and is connected to third water supply pipe 222. When operating in response to an instruction from controller 500, third pump 220 pumps up electrolyzed water stored in electrolysis tank 200 toward third water supply pipe 222. Third water supply pipe 222 is connected to fixed capacity container 224, and supplies the electrolyzed water in electrolysis tank 200 to fixed capacity container 224. Fixed capacity container 224 is a measuring container having a fixed volume, and stores a fixed volume of electrolyzed water supplied from third water supply pipe 222. Fixed capacity container 224 is connected to third water supply pipe 226, and third water supply pipe 226 extends toward humidification tank 300. Electrolyzed water stored in fixed capacity container 224 flows in third water supply pipe 226 and is supplied to humidification tank 300. That is, third pump 220, third water supply pipe 222, fixed capacity container 224, and third water supply pipe 226 supply electrolyzed water from electrolysis tank 200 to humidification tank 300.

Humidification tank 300 has a box shape with a top surface opened, and mixes the water supplied from water storage tank 100 and the electrolyzed water supplied from electrolysis tank 200. This corresponds to diluting electrolyzed water supplied from electrolysis tank 200 with water supplied from water storage tank 100. Humidification tank 300 is provided with cleaner 310.

Cleaner 310 includes a fan (not illustrated) and a filter (filter). The fan is, for example, a sirocco fan, and rotates under the control of controller 500. As the fan rotates, air is sucked into space cleaning device 1000 from an intake port (not illustrated) provided in a housing (not illustrated) of space cleaning device 1000.

The filter is a member that brings the electrolyzed water stored in humidification tank 300 into contact with the indoor air having flowed into space cleaning device 1000 by the fan. The filter is formed in a cylindrical shape, and has a hole through which air can flow in a circumferential portion. The filter is rotatably incorporated in humidification tank 300 with a central axis as a rotation center so that one end of the filter is immersed in the electrolyzed water stored in humidification tank 300 to retain water. The filter is rotated by a driver (not illustrated) to bring the electrolyzed water and the indoor air into continuous contact with each other.

An air path leading from the intake port to the filter, the fan, and a blow-out port (not illustrated) is formed. When the fan rotates, the external air having been sucked from the intake port and having entered the air path is sequentially blown out to the outside of space cleaning device 1000 via the filter, the fan, and the blow-out port. Due to this, the air brought into contact with the electrolyzed water in humidification tank 300 is released to the outside. Space cleaning device 1000 releases active oxygen species derived from the electrolyzed water having been generated (including volatilization) together with air.

Each of water shortage float 160 provided in water storage tank 100, full water float 250 and water shortage float 260 provided in electrolysis tank 200, and full water float 350, water shortage float 360, and drainage float 370 provided in humidification tank 300 detects whether or not water or electrolyzed water is present. Here, water and electrolyzed water may be collectively referred to as “water”. Water shortage float 160, full water float 250, water shortage float 260, full water float 350, water shortage float 360, and drainage float 370 are collectively referred to as “float”. Each float has buoyancy and a magnet (not illustrated), and the position of the magnet is detected by a detection portion (not illustrated). In a case where water is present up to the position of the float, the float moves to a predetermined position by buoyancy, and the detection portion detects the magnet provided on the float portion. On the other hand, in a case where water is not present up to the position of the float, the detection portion cannot detect the magnet provided on the float.

Water shortage float 160 detects a water shortage of water storage tank 100. Full water float 250 detects full water of electrolysis tank 200, and water shortage float 260 detects a water shortage of electrolysis tank 200. Here, the water shortage does not have to be a 100% water shortage, and a slight amount of water may remain. In the present exemplary embodiment, water shortage float 260 may be referred to as water shortage detector. Full water float 350 detects full water of humidification tank 300, water shortage float 360 detects a water shortage of humidification tank 300, and drainage float 370 detects the drainage level of humidification tank 300. Here, the full water does not have to be 100% full water, and may be a water amount at which water can be further put. Each float transmits a detection result to controller 500.

Controller 500 receives detection results from water shortage float 160, full water float 250, water shortage float 260, full water float 350, water shortage float 360, and drainage float 370. Controller 500 executes control of electrodes 210, cleaner 310, electrolysis accelerator inputter 400, first supplier 128, second supplier 138, and third supplier 228. Details of the process of controller 500 will be described later.

As an example, the concentration of electrolyzed water to be generated in electrolysis tank 200 is a concentration within a range of 30 ppm to 200 ppm (hereinafter, referred to as “first concentration”), and the concentration of electrolyzed water to be diluted in humidification tank 300 is a concentration within a range of 3 ppm to 50 ppm. The concentration of the electrolyzed water to be diluted in humidification tank 300 is set to be lower than the concentration of the electrolyzed water to be generated in electrolysis tank 200.

### (2) Initial Process

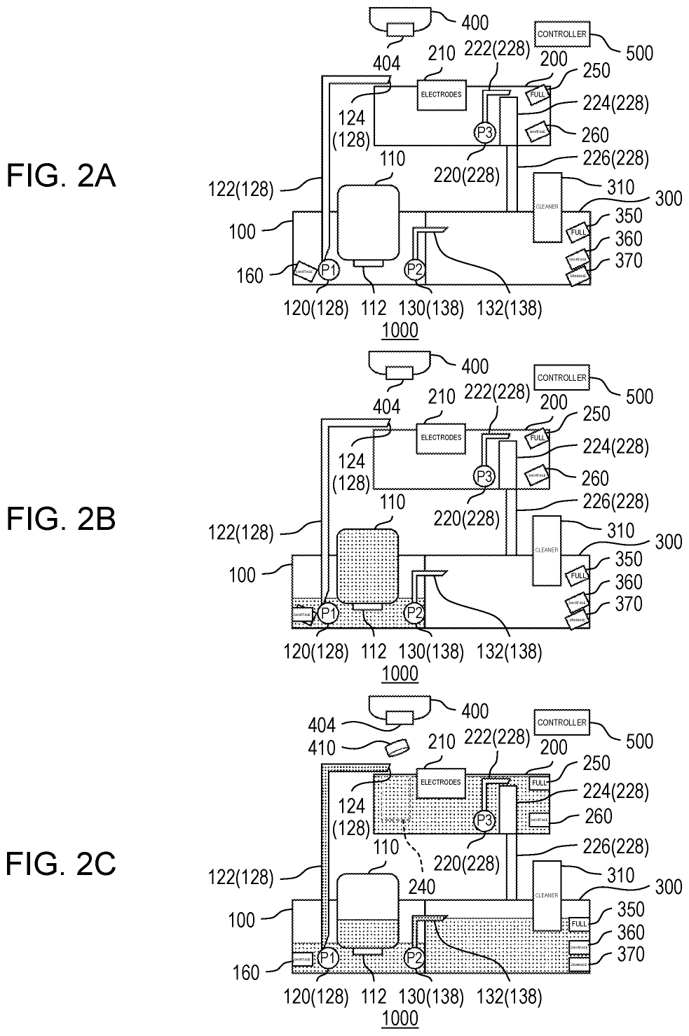

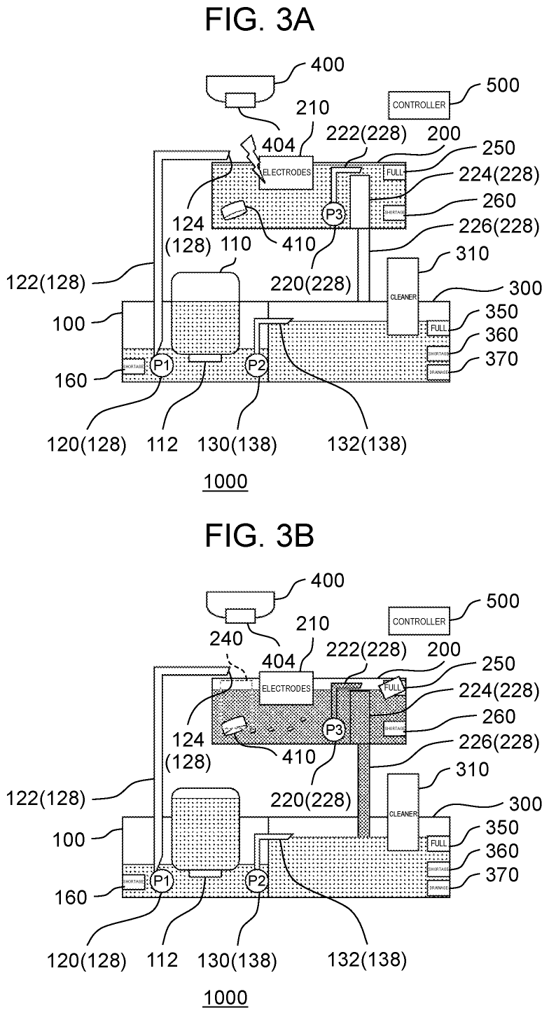

The initial process is a process from a state in which there is no water in water storage tank 100, electrolysis tank 200, and humidification tank 300 to execution of release of the electrolyzed water in an initial stage. In the following, the initial process will be described also with reference to FIGS. 2A to 2C, 3A, and 3B. FIGS. 2A to 2C illustrate an operation outline of space cleaning device 1000.

FIG. 2A illustrates a state where there is no water in water storage tank 100, electrolysis tank 200, and humidification tank 300. This corresponds to a case where space cleaning device 1000 is installed after space cleaning device 1000 is purchased. This also corresponds to a case after maintenance such as cleaning of water storage tank 100, electrolysis tank 200, and the humidification tank 300.

FIG. 2B illustrates a state following FIG. 2A. The user pours water into water supply tank 110, and attaches water supply tank 110 to water storage tank 100. When water supply tank 110 is attached to water storage tank 100, water is supplied from water supply tank 110 to water storage tank 100 by opening the opening and closing portion of lid 112.

FIG. 2C illustrates a state following FIG. 2B. By operating second pump 130, controller 500 supplies the water in water storage tank 100 to humidification tank 300. The water is supplied until full water float 350 detects full water. As a result, humidification tank 300 stores water in a state of being full water.

By operating first pump 120, controller 500 supplies water in water storage tank 100 to electrolysis tank 200. At that time, water is supplied for a certain period of time for which electrolysis tank 200 is not filled to full water. As a result of the water supply, the water surface of electrolysis tank 200 is at a water level lower than the water level of full water. Supply region 240 is disposed on a part of the water surface of electrolysis tank 200, and supply region 240 is located at the lower side of supply port 124 and input port 404. After the water supply is completed, controller 500 causes electrolysis accelerator 410 to be dropped from input port 404 toward supply region 240 of electrolysis tank 200. As a result, electrolysis accelerator 410 is present in supply region 240 and begins to dissolve in water.

Subsequently, by operating first pump 120 again, controller 500 supplies water in water storage tank 100 to electrolysis tank 200. At this time, since water is supplied from supply port 124 toward supply region 240, the dissolution of electrolysis accelerator 410 further proceeds by the pressure of the supplied water. The water is supplied until full water float 250 detects full water. As a result, humidification tank 300 stores water containing chloride ions in which some or all of electrolysis accelerators 410 are dissolved in a state of being full water.

FIGS. 3A and 3B illustrate an operation outline of space cleaning device 1000 following FIGS. 2A to 2C.

FIG. 3A illustrates a state following FIG. 2C. By executing electric conduction to electrodes 210, controller 500 electrolyzes water containing chloride ions to generate electrolyzed water. Here, the electrolysis time is set to a time (e.g., 10 minutes) shorter than a time (e.g., 40 minutes) required for generating the electrolyzed water having the first concentration. As a result, electrolyzed water having the second concentration lower than the first concentration is generated.

FIG. 3B illustrates a state following FIG. 3A. When the electrolyzed water having the second concentration is generated, controller 500 supplies the electrolyzed water having the second concentration to humidification tank 300 by operating third pump 220. At this time, since fixed capacity container 224 is used, the electrolyzed water having the second concentration corresponding to the volume of fixed capacity container 224 is supplied to humidification tank 300. Electrolyzed water having the second concentration is diluted in humidification tank 300. By operating cleaner 310 after stopping third pump 220, controller 500 releases the air brought into contact with the electrolyzed water in humidification tank 300 to the outside of space cleaning device 1000. That is, release of the air brought into contact with the electrolyzed water is started in a time shorter than 40 minutes.

### (3) Normal Process

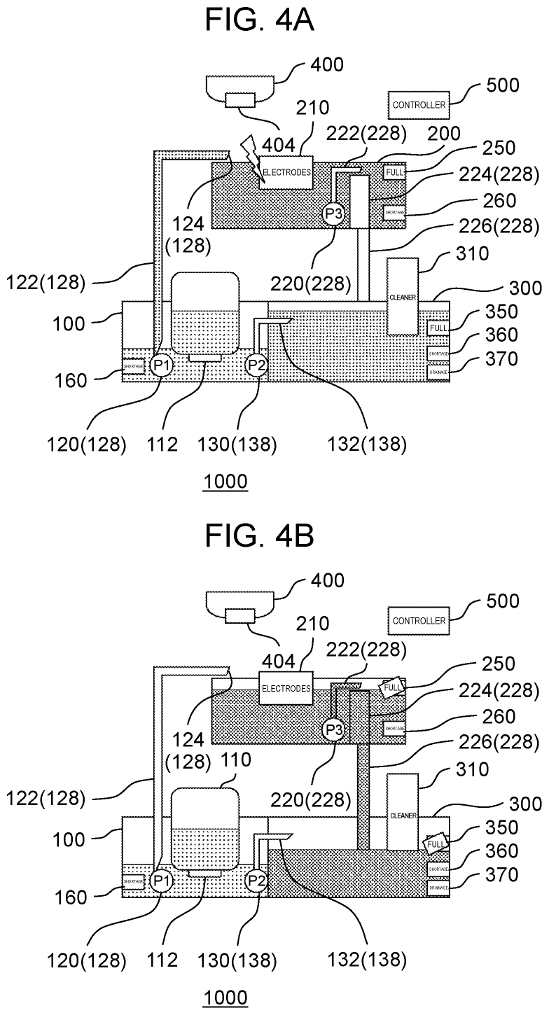

The normal process is a process for releasing electrolyzed water of a desired concentration. FIGS. 4A and 4B illustrate an operation outline of space cleaning device 1000 following FIGS. 3A and 3B.

FIG. 4A illustrates a state following FIG. 3B. Since a part of the electrolyzed water having the second concentration in electrolysis tank 200 has been supplied to humidification tank 300, the electrolyzed water having the second concentration is stored in electrolysis tank 200 in a state of not being full water. By operating first pump 120, controller 500 supplies water in water storage tank 100 to electrolysis tank 200. At this time, since water is supplied from supply port 124 toward supply region 240, remaining electrolysis accelerator 410 is further dissolved by the pressure of the supplied water. The water is supplied until full water float 250 detects full water. As a result, electrolysis tank 200 is brought into a state of being full water. After the water supply to electrolysis tank 200 has ended, controller 500 generates electrolyzed water by electrolysis by executing electric conduction to electrodes 210. Here, the electrolysis time is a time (e.g., 40 minutes) required for generating the electrolyzed water having the first concentration. As a result, electrolyzed water having the first concentration is generated.

FIG. 4B illustrates a state following FIG. 4A. When the electrolyzed water having the first concentration is generated, controller 500 supplies the electrolyzed water having the first concentration to humidification tank 300 by operating third pump 220. At this time, since fixed capacity container 224 is used, the electrolyzed water having the first concentration corresponding to the volume of fixed capacity container 224 is supplied to humidification tank 300. Electrolyzed water having the first concentration is diluted in humidification tank 300. By operating cleaner 310 after stopping third pump 220, controller 500 releases the air brought into contact with the electrolyzed water in humidification tank 300 to the outside of space cleaning device 1000.

When the air brought into contact with the electrolyzed water is released, the amount of the electrolyzed water in humidification tank 300 decreases. When water shortage float 360 detects a water shortage, controller 500 supplies the electrolyzed water having the first concentration to humidification tank 300 by the volume of fixed capacity container 224 by operating third pump 220, and by operating second pump 130, supplies the water in water storage tank 100 until humidification tank 300 becomes full. Due to this, release of the electrolyzed water is continued. Such process is repeated until water shortage float 260 detects a water shortage.

### (4) Reprocessing

Reprocessing is a process for executing the normal process again when water shortage float 260 detects a water shortage, that is, when the electrolyzed water in electrolysis tank 200 becomes short of water. When water shortage float 260 detects a water shortage after the electrolyzed water having the first concentration is supplied to humidification tank 300, controller 500 starts supplying water to electrolysis tank 200 by first supplier 128. That is, controller 500 does not supply water to electrolysis tank 200 until electrolysis tank 200 becomes short of water. This is because the concentration of the electrolyzed water in electrolysis tank 200 is maintained at the first concentration by not supplying water. This is because impurities such as inorganic salt compounds are less likely to remain in electrolysis tank 200 by making old electrolyzed water less likely to remain in electrolysis tank 200. This reduces the maintenance frequency of electrolysis tank 200.

Here, same as the initial process, controller 500 executes water supply for a certain period of time for which electrolysis tank 200 is not filled to full water. Subsequently, controller 500 causes electrolysis accelerator 410 to be dropped from input port 404 toward supply region 240 of electrolysis tank 200, and executes water supply until electrolysis tank 200 is filled with water. By executing electric conduction to electrodes 210, controller 500 generates electrolyzed water having the second concentration, and then supplies the electrolyzed water having the second concentration from electrolysis tank 200 to humidification tank 300. That is, the same process as a part of the initial process is executed. Subsequently, normal process is executed.

### (5) Input Process of Electrolysis Accelerator

As mentioned earlier, electrolysis accelerator inputter 400 inputs electrolysis accelerator 410 into electrolysis tank 200. Here, the configuration and operation related to input of electrolysis accelerator 410 will be described.

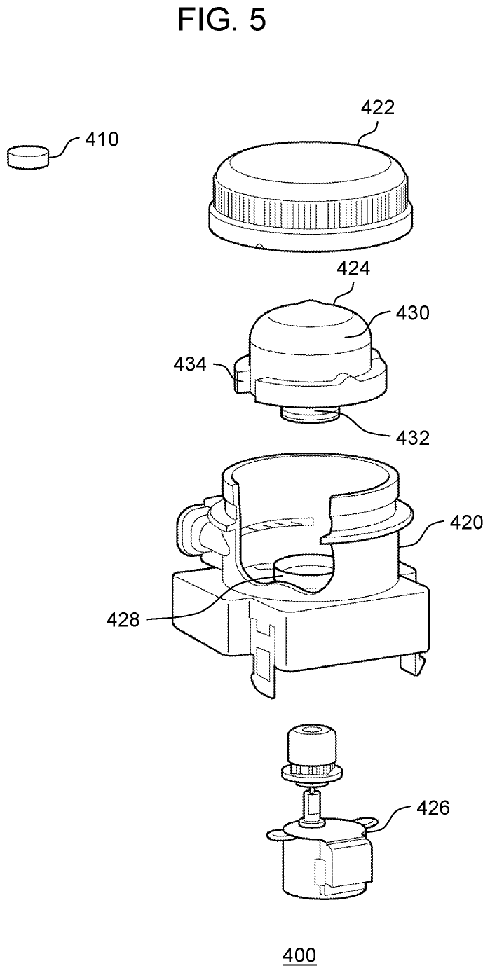

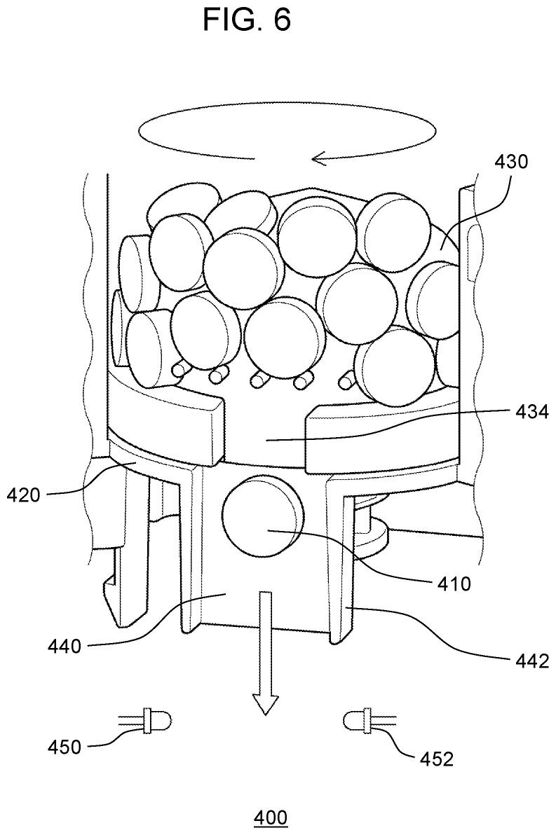

FIG. 5 is an exploded perspective view of electrolysis accelerator inputter 400, and is a view in which a part of case 420 is cut out so that the inside of case 420 is visible. FIG. 6 is a perspective view illustrating the inside of case 420 of electrolysis accelerator inputter 400, particularly an enlarged perspective view of hole 440 of case 420 and cutout 434 of rotator 424 of electrolysis accelerator inputter 400, and is a view illustrating a scene where electrolysis accelerator 410 drops through cutout 434 of rotator 424 and hole 440 of case 420.

As illustrated in FIGS. 5 and 6, electrolysis accelerator inputter 400 includes case 420, case cover 422, rotator 424, motor 426, light emitter 450, and light receiver 452. Case 420 has a circular deep dish shape with the upper side opened. Case cover 422 having a bowl shape with the lower side opened is detachable from the upper end of case 420. Case 420 internally includes rotator 424, and includes motor 426 lower in the case. Motor 426 rotates rotator 424 in case 420 with the vertical direction as a rotation shaft direction. Rotator 424 corresponds to the tablet input member mentioned earlier. Bearing hole 428 and hole 440 are disposed on the bottom surface of case 420. Bearing hole 428 is a hole into which rotation shaft 432 of rotator 424 described later enters. Hole 440 is a hole through which electrolysis accelerator 410 passes. Guide tube 442 extending downward from an opening edge of hole 440 is disposed in hole 440.

Light emitter 450 is provided on the side of hole 440 through which electrolysis accelerator 410 passes and guide tube 442, and in perpendicular to the passing direction of electrolysis accelerator 410. Light emitter 450 includes, for example, an infrared light emitting diode (LED), and is disposed so as to emit light toward a passing position of electrolysis accelerator 410.

Light receiver 452 is disposed on the side of hole 440 through which electrolysis accelerator 410 passes and guide tube 442 and at a position facing light emitter 450 across hole 440. Light receiver 452 is disposed with the light receiving surface facing the passing position of electrolysis accelerator 410, and can receive light of light emitter 450. Upon receiving light from light emitter 450, light receiver 452 outputs a signal according to the intensity of the received light. As one aspect of an output signal of light receiver 452, the intensity of the signal decreases, for example, when the light is blocked by passage of electrolysis accelerator 410, that is, the intensity of the received light decreases.

Rotator 424 has convex surface 430 whose center is a disk having an upward convex shape, and rotation shaft 432 having a cylindrical shape extending downward from a center lower surface of convex surface 430. Convex surface 430 and rotation shaft 432 are integrally formed of a resin material. Convex surface 430 is slightly smaller in size than case 420, and has a slight gap between the outer periphery of convex surface 430 and the inner surface of case 420. Cutout 434 is disposed in a peripheral edge of convex surface 430. When electrolysis accelerator 410 enters cutout 434 and overlaps hole 440, electrolysis accelerator 410 is input into electrolysis tank 200.

In such configuration, even when controller 500 instructs electrolysis accelerator inputter 400 to input electrolysis accelerator 410, the intensity of the light received by light receiver 452 does not decrease, that is, the passage of electrolysis accelerator 410 is not detected on rare occasions. Such situation occurs due to the following two causes. The first cause is a case where electrolysis accelerator 410 passes through cutout 434 and hole 440 and is dropped into electrolysis tank 200, but a decrease in intensity of the light received by light receiver 452 is small. This is erroneous detection by light receiver 452. The second cause is a case where electrolysis accelerator 410 does not pass through cutout 434 and hole 440 and is not input into electrolysis tank 200 even if cutout 434 and hole 440 overlap with each other due to rotation of rotator 424. This occurs, for example, when electrolysis accelerator 410 is about to be dissolved and electrolysis accelerator 410 is adhered to the wall surface of cutout 434. An occasion where the passage of electrolysis accelerator 410 is not detected occurs also when electrolysis accelerator 410 in electrolysis accelerator inputter 400 runs out of due to repeated input of electrolysis accelerator 410 by electrolysis accelerator inputter 400.

As mentioned earlier, in such situation, an instruction to input electrolysis accelerator 410 is given again. As a result, particularly in the case of the first cause, more than a predetermined number of electrolysis accelerators 410 are input, and electrolyzed water having the concentration of hypochlorous acid higher than a reference value is generated. In order to suppress the concentration of hypochlorous acid in electrolyzed water from becoming higher than a reference value, space cleaning device 1000 according to the present exemplary embodiment executes the following process.

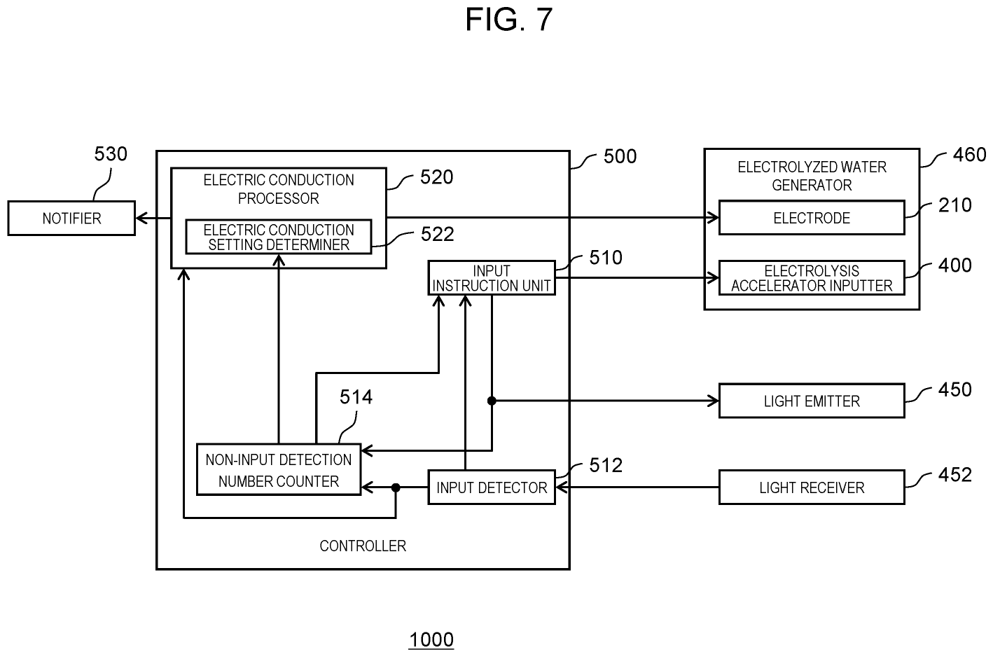

FIG. 7 illustrates functional blocks of space cleaning device 1000. Space cleaning device 1000 includes light emitter 450, light receiver 452, electrolyzed water generator 460, controller 500, and notifier 530. Electrolyzed water generator 460 includes electrodes 210 and electrolysis accelerator inputter 400. Controller 500 includes input instruction unit 510, input detector 512, non-input detection number counter 514, and electric conduction processor 520, and electric conduction processor 520 includes electric conduction setting determiner 522.

Input instruction unit 510 instructs electrolysis accelerator inputter 400 to input electrolysis accelerator 410. The timing at which input instruction unit 510 instructs the input of electrolysis accelerator 410 is as mentioned earlier. Input instruction unit 510 instructs the input of electrolysis accelerator 410 and instructs light emitter 450 to emit light, and notifies non-input detection number counter 514 of an instruction to input electrolysis accelerator 410.

Input detector 512 detects whether or not electrolysis accelerator 410 has passed through hole 440 based on a signal of light receiver 452, that is, whether or not electrolysis accelerator 410 has been input. For example, input detector 512 determines that electrolysis accelerator 410 has passed through hole 440 when the intensity of the received light is lower than a threshold, and determines that electrolysis accelerator 410 has not passed through hole 440 when the intensity of the received light is equal to or greater than the threshold. The threshold is set in advance through, for example, simulation, experiment, or the like. Therefore, “based on a signal of light receiver 452” mentioned earlier means attenuation of the signal, that is, a decrease in the intensity of light. Input detector 512 outputs the detection result to input instruction unit 510, non-input detection number counter 514, and electric conduction processor 520.

When the detection result received from input detector 512 does not indicate the input of electrolysis accelerator 410, input instruction unit 510 repeats the process so far. Specifically, input instruction unit 510 instructs electrolysis accelerator inputter 400 to input electrolysis accelerator 410, instructs light emitter 450 to emit light, and notifies non-input detection number counter 514 of the instruction to input electrolysis accelerator 410. Such process is repeated until input detector 512 detects input of electrolysis accelerator 410. As described later, an upper limit value may be provided for the number of times of repetition.

Non-input detection number counter 514 receives, from input instruction unit 510, an instruction to input electrolysis accelerator 410, and receives a detection result from input detector 512. Non-input detection number counter 514 counts the number of times input detector 512 has not detected the input of electrolysis accelerator 410 although input instruction unit 510 has instructed the input of electrolysis accelerator 410. The number of times is the “non-input detection number”. The non-input detection number is reset when the detection result received from input detector 512 indicates the input of electrolysis accelerator 410 or when the number of times of repetition of the input instruction by input instruction unit 510 reaches the upper limit value.

Electric conduction setting determiner 522 receives the non-input detection number from non-input detection number counter 514. Electric conduction setting determiner 522 determines the electric conduction setting for the electrolysis in electrolyzed water generator 460 based on the non-input detection number. For example, the electric conduction setting is setting of the electric conduction time, and electric conduction setting determiner 522 shortens the electric conduction time as the non-input detection number increases. More specifically, electric conduction setting determiner 522 determines the electric conduction time by a specified value of the electric conduction time (hereinafter, referred to as “specified time”)/(1+non-input detection number). An example of the specified time is “40 minutes” mentioned earlier. Therefore, when input detector 512 does not fail to detect input, the electric conduction time is a specified time.

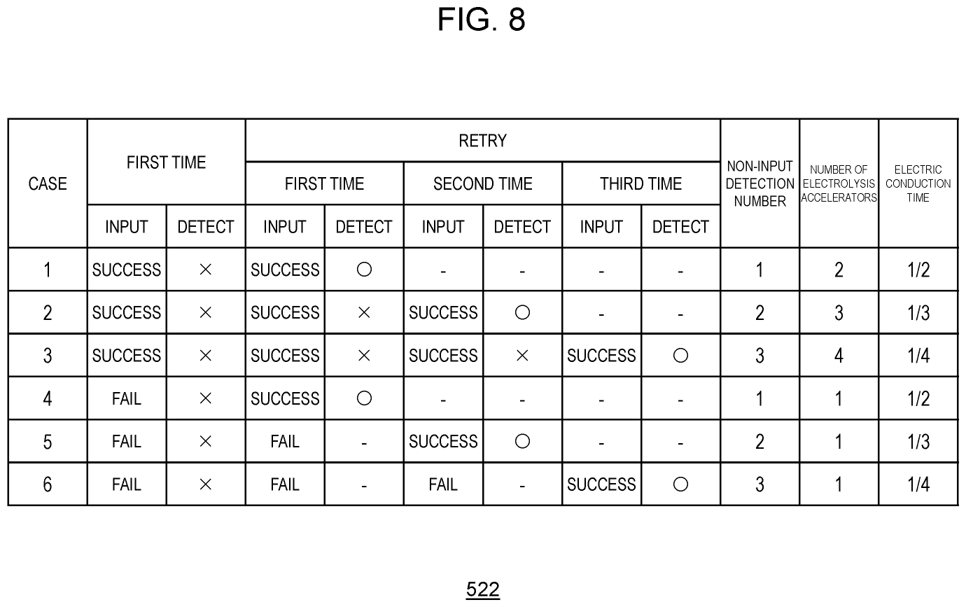

FIG. 8 illustrates an outline of the process in electric conduction setting determiner 522. Here, “1” to “6” are assumed as cases. Case “1” to case “3” correspond to the first cause mentioned earlier. Case “4” to case “6” correspond to the second cause mentioned earlier. In the case “1”, when input instruction unit 510 instructs the first input, electrolysis accelerator inputter 400 succeeds in input, but input detector 512 fails in detection. Subsequently, when input instruction unit 510 instructs input of the first retry, electrolysis accelerator inputter 400 succeeds in input, and input detector 512 also succeeds in detection. As a result, the non-input detection number is “1”, and the number of electrolysis accelerators 410 having been input is “2”. Electric conduction setting determiner 522 determines the electric conduction time to be “½” of the specified time.

In the case “2”, when input instruction unit 510 instructs the first input, electrolysis accelerator inputter 400 succeeds in input, but input detector 512 fails in detection. The same applies to the first retry. When input instruction unit 510 instructs input of the second retry, electrolysis accelerator inputter 400 succeeds in input, and input detector 512 also succeeds in detection. As a result, the non-input detection number is “2”, and the number of electrolysis accelerators 410 having been input is “3”. Electric conduction setting determiner 522 determines the electric conduction time to be “⅓” of the specified time.

In the case “3”, when input instruction unit 510 instructs the first input, electrolysis accelerator inputter 400 succeeds in input, but input detector 512 fails in detection. The same applies to the first and second retries. When input instruction unit 510 instructs input of the third retry, electrolysis accelerator inputter 400 succeeds in input, and input detector 512 also succeeds in detection. As a result, the non-input detection number is “3”, and the number of electrolysis accelerators 410 having been input is “4”. Electric conduction setting determiner 522 determines the electric conduction time to be “¼” of the specified time.

In the case “4”, when input instruction unit 510 instructs the first input, electrolysis accelerator inputter 400 fails in input, and input detector 512 fails in the detection. Subsequently, when input instruction unit 510 instructs input of the first retry, electrolysis accelerator inputter 400 succeeds in input, and input detector 512 also succeeds in detection. As a result, the non-input detection number is “1”, and the number of electrolysis accelerators 410 having been input is “1”. Electric conduction setting determiner 522 determines the electric conduction time to be “½” of the specified time.

In the case “5”, when input instruction unit 510 instructs the first input, electrolysis accelerator inputter 400 fails in input, and input detector 512 fails in the detection. The same applies to the first retry. When input instruction unit 510 instructs input of the second retry, electrolysis accelerator inputter 400 succeeds in input, and input detector 512 also succeeds in detection. As a result, the non-input detection number is “2”, and the number of electrolysis accelerators 410 having been input is “1”. Electric conduction setting determiner 522 determines the electric conduction time to be “⅓” of the specified time.

In the case “6”, when input instruction unit 510 instructs the first input, electrolysis accelerator inputter 400 fails in input, and input detector 512 fails in the detection. The same applies to the first and second retries. When input instruction unit 510 instructs input of the third retry, electrolysis accelerator inputter 400 succeeds in input, and input detector 512 also succeeds in detection. As a result, the non-input detection number is “3”, and the number of electrolysis accelerators 410 having been input is “1”. Electric conduction setting determiner 522 determines the electric conduction time to be “¼” of the specified time.

In cases “1” to “3”, the number of electrolysis accelerators 410 having been input is larger than “1”, which is a predetermined number. However, since the electric conduction time is made shorter than the specified time, the concentration of hypochlorous acid in the electrolyzed water is suppressed from becoming higher than a reference value. On the other hand, in cases “4” to “6”, the number of electrolysis accelerators 410 having been input is “1”, which is the predetermined number. Furthermore, since the electric conduction time is made shorter than the specified time, the concentration of hypochlorous acid in the electrolyzed water is lowered, but the concentration is not higher than the reference value, and thus safety is secured.

After input detector 512 detects the input of electrolysis accelerator 410, electric conduction processor 520 executes electric conduction to electrodes 210 according to the electric conduction time set by electric conduction setting determiner 522. In response to this, electrolyzed water generator 460 generates electrolyzed water by electric conduction setting after input detector 512 detects the input of electrolysis accelerator 410.

When the non-input detection number counted by non-input detection number counter 514 becomes equal to or greater than a threshold, input instruction unit 510 does not instruct input of electrolysis accelerator 410 by electrolysis accelerator inputter 400. The threshold is, for example, “4”. When the non-input detection number becomes equal to or greater than the threshold, notifier 530 notifies an input error by electrolysis accelerator inputter 400.

The devices, the systems, or the subject of methods in the present disclosure include a computer. This computer executes a program, thereby implementing the function of the devices, the systems, or the subject of methods in the present disclosure. The computer includes, as a main hardware configuration, a processor that operates according to a program. The type of the processor is not limited as long as the processor can implement the function by executing a program. The processor includes one or more electronic circuits including a semiconductor integrated circuit or a large scale integration (LSI). The plurality of electronic circuits may be integrated on one chip or may be provided on a plurality of chips. The plurality of chips may be aggregated into one device or may be provided in a plurality of devices. The program is recorded in a computer-readable non-transitory recording medium such as a read only memory (ROM), an optical disk, or a hard disk drive. The program may be stored in advance in a recording medium, or may be supplied to the recording medium via a wide area communication network including the Internet and the like.

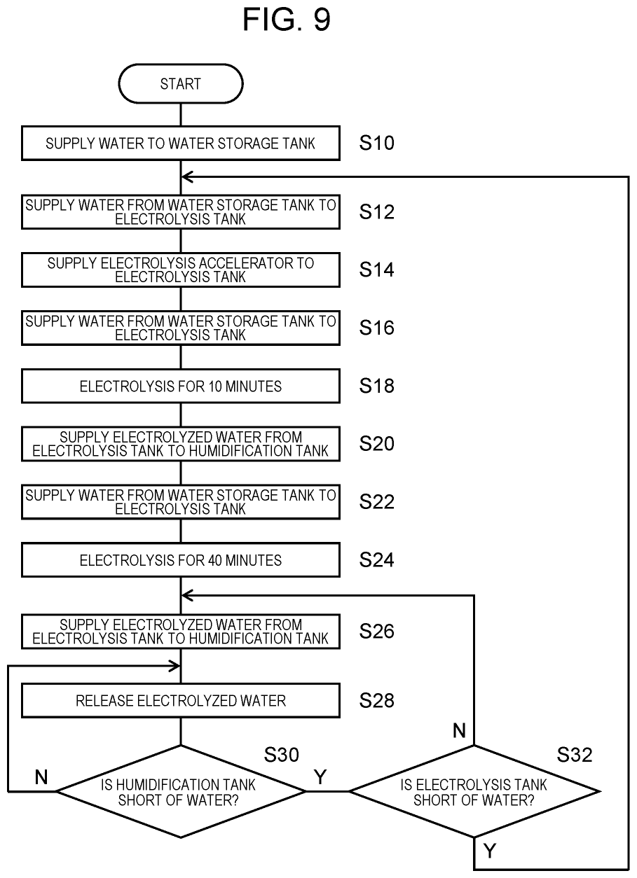

The operation of space cleaning device 1000 having the above configuration will be described. FIG. 9 is a flowchart showing a control procedure by space cleaning device 1000.

First, water is supplied from the outside to water storage tank 100 (S10).

Next, water is supplied from water storage tank 100 to electrolysis tank 200 by an amount smaller than the full water in electrolysis tank 200 (S12).

Next, electrolysis accelerator 410 is supplied from electrolysis accelerator inputter 400 to electrolysis tank 200 (S14).

Next, water is supplied from water storage tank 100 to electrolysis tank 200 until electrolysis tank 200 is filled with water (S16).

Next, electrodes 210 execute electrolysis for 10 minutes (S18). Due to this, electrolyzed water having the second concentration is generated in electrolysis tank 200.

Next, electrolyzed water having the second concentration is supplied from electrolysis tank 200 to humidification tank 300 (S20). Due to this, electrolyzed water is released in humidification tank 300.

Next, water is supplied from water storage tank 100 to electrolysis tank 200 until electrolysis tank 200 is filled with water (S22).

Next, electrodes 210 execute electrolysis for 40 minutes (S24). Due to this, electrolyzed water having the first concentration is generated in electrolysis tank 200.

Next, electrolyzed water having the first concentration is supplied from electrolysis tank 200 to humidification tank 300 (S26).

Next, cleaner 310 releases the electrolyzed water (S28).

Next, water shortage float 360 determines whether or not humidification tank 300 is in short of water (S30). When it is determined in step S30 that humidification tank 300 is not in short of water (N in S30), the process returns to step S28.

When it is determined in step S30 that humidification tank 300 is in short of water (Y in S30), water shortage float 260 determines whether or not electrolysis tank 200 is in short of water (S32). When it is determined in step S32 that electrolysis tank 200 is not in short of water (N in S32), the process returns to step S26.

When it is determined in step S32 that electrolysis tank 200 is in short of water (Y in S32), the process returns to step S12.

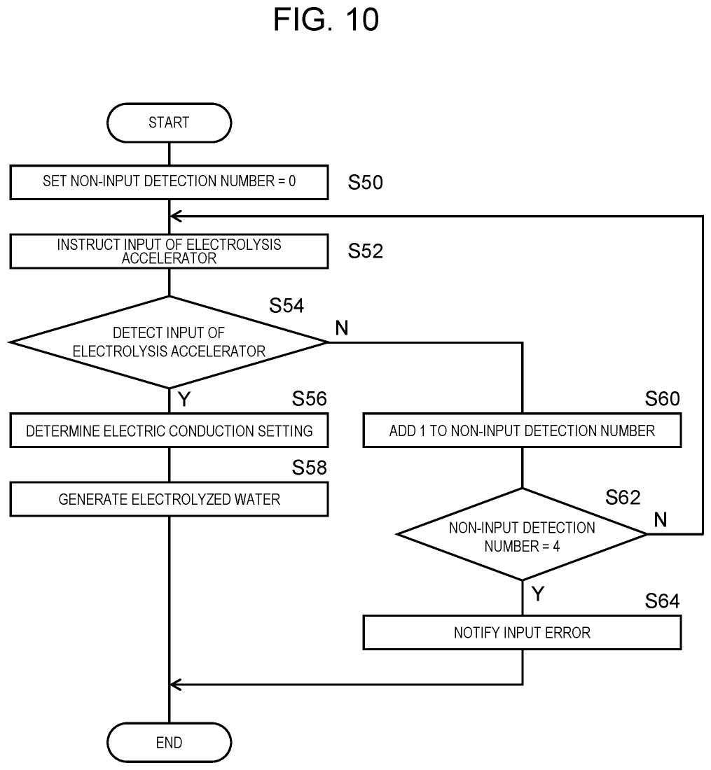

FIG. 10 is a flowchart showing an electric conduction procedure by space cleaning device 1000.

First, non-input detection number counter 514 sets the non-input detection number=0 (S50).

Next, input instruction unit 510 instructs electrolysis accelerator inputter 400 to input electrolysis accelerator 410 (S52).

Next, it is determined whether or not input detector 512 has detected input of electrolysis accelerator 410 (S54). When input detector 512 detects the input of electrolysis accelerator 410 in step S54 (Y in S54), electric conduction setting determiner 522 determines the electric conduction setting based on the non-input detection number (S56). Thereafter, electrolyzed water generator 460 generates electrolyzed water according to the electric conduction setting (S58).

When input detector 512 does not detect the input of electrolysis accelerator 410 in step S54 (N in S54), non-input detection number counter 514 adds “1” to the non-input detection number (S60). Thereafter, non-input detection number counter 514 determines whether or not the non-input detection number is “4” (S62).

When it is determined in step S62 that the non-input detection number is not “4” (N in S62), the process returns to step S52.

When it is determined in step S62 that the non-input detection number is “4” (Y in S62), notifier 530 notifies an input error (S64).

According to the present exemplary embodiment, since the electric conduction setting is determined based on the non-input detection number having been counted, it is possible to determine the electric conduction setting including erroneous detection even if erroneous detection occurs. Since electric conduction setting including erroneous detection is determined even if erroneous detection occurs, it is possible to suppress the concentration of hypochlorous acid in electrolyzed water from becoming higher than a reference value. Since the concentration of hypochlorous acid in electrolyzed water is suppressed from becoming higher than a reference value, safety can be secured. Since input of electrolysis accelerator 410 is instructed until the input of electrolysis accelerator 410 is detected, and after the input of electrolysis accelerator 410 is detected, electrolyzed water is generated by the electric conduction setting, electrolyzed water can be reliably generated.

Since the electric conduction time is shortened as the non-input detection number increases, it is possible to suppress a rise in the concentration of hypochlorous acid in the electrolyzed water even when a large amount of electrolysis accelerator 410 is input. Since when the non-input detection number becomes equal to or greater than the threshold, input of electrolysis accelerator 410 is not instructed, the operation of space cleaning device 1000 can be stopped when there is a risk of a malfunction of space cleaning device 1000. Since the input error is notified when the non-input detection number becomes equal to or greater than the threshold, the occurrence of a trouble can be notified. Since the input error is notified when the non-input detection number becomes equal to or greater than the threshold, it is possible to notify that electrolysis accelerator 410 in electrolysis accelerator inputter 400 has run out.

Since the water process tank is divided into water storage tank 100, electrolysis tank 200, and humidification tank 300, it is possible to suppress an occurrence of gas-liquid contact with water in electrolysis tank 200 used by electrodes 210. Since the occurrence of gas-liquid contact with water in electrolysis tank 200 is suppressed, the water in electrolysis tank 200 can be made less likely to be contaminated. Since the water in electrolysis tank 200 is less likely to be contaminated, deterioration of an electrodes can be suppressed. Since the electrolyzed water having the second concentration is supplied to humidification tank 300 and released, the period until the electrolyzed water is released can be shortened. Since following the electrolyzed water having the second concentration, the electrolyzed water having the first concentration is generated, electrolyzed water having a desired concentration can be released. Since electrolysis accelerator 410 is input toward supply region 240 and water is supplied toward supply region 240, the dissolution of electrolysis accelerator 410 can be proceeded by the pressure of water. Since the electrolyzed water having the first concentration is generated by the normal process after the water is supplied to electrolysis tank 200, electrolysis accelerator 410 can be easily dissolved.

When water shortage is detected, water is supplied to electrolysis tank 200 by first supplier 128, making the water supply unnecessary until the water shortage is detected. Since the water supply becomes unnecessary until the water shortage is detected, the concentration of the electrolyzed water in electrolysis tank 200 can be maintained. Since the water supply becomes unnecessary until the water shortage is detected, the impurities remaining in electrolysis tank 200 can be flowed. Since a part of the initial process is executed as the reprocessing, the operation can be simplified.

An outline of one aspect of the present disclosure is as follows. Space cleaning device (1000) of an aspect of the present disclosure includes: electrolysis tank (200) that stores water and electrolyzed water; an electrolysis accelerator inputter (400) that inputs electrolysis accelerator (410) into electrolysis tank (200); electrolyzed water generator (460) that generates the electrolyzed water by electrolyzing the water into which electrolysis accelerator (410) is input; cleaner (310) that brings the electrolyzed water generated by electrolyzed water generator (460) into contact with air sucked from an intake port; input instruction unit (510) that instructs electrolysis accelerator inputter (400) to input electrolysis accelerator (410); input detector (512) that detects input of electrolysis accelerator (410) by electrolysis accelerator inputter (400); non-input detection number counter (514) that counts a non-input detection number that is a number of times input detector (512) has not detected input of electrolysis accelerator (410) even after input instruction unit (510) has instructed the input of electrolysis accelerator (410); and electric conduction setting determiner (522) that determines electric conduction setting for electrolysis in electrolyzed water generator (460) based on the non-input detection number counted by non-input detection number counter (514).

Input instruction unit (510) may instruct electrolysis accelerator inputter (400) to input electrolysis accelerator (410) until input detector (512) detects the input of electrolysis accelerator (410), and electrolyzed water generator (460) may generate the electrolyzed water by the electric conduction setting after input detector (512) detects the input of electrolysis accelerator (410).

Electric conduction setting may include setting of an electric conduction time in electrolyzed water generator (460). Electric conduction setting determiner (522) may shorten the electric conduction time as the non-input detection number increases.

Electric conduction setting may include setting of an electric conduction current value in electrolyzed water generator (460). Electric conduction setting determiner (522) may shorten the electric conduction current value as the non-input detection number increases.

Electric conduction setting may include setting of an electric conduction voltage value in electrolyzed water generator (460). Electric conduction setting determiner (522) may shorten the electric conduction voltage value as the non-input detection number increases.

When the non-input detection number becomes equal to or greater than the threshold, input instruction unit (510) does not need to instruct the input of electrolysis accelerator (410) by electrolysis accelerator inputter (400).

Notifier (530) that notifies an input error by electrolysis accelerator inputter (400) when the non-input detection number becomes equal to or greater than the threshold may further be included.

The present disclosure has been described above based on the exemplary embodiment. It will be understood by those skilled in the art that the exemplary embodiment is merely an example, various modifications in combinations of components or processes of the exemplary embodiment are possible, and such modifications fall within the scope of the present disclosure.

Electric conduction setting determiner 522 in the present exemplary embodiment sets the electric conduction time as the electric conduction setting. However, the present disclosure is not limited to this, and for example, electric conduction setting determiner 522 may set the electric conduction current value as the electric conduction setting. At that time, electric conduction setting determiner 522 decreases the electric conduction current value as the non-input detection number increases. Electric conduction setting determiner 522 may set the electric conduction voltage value as the electric conduction setting. At that time, electric conduction setting determiner 522 decreases the electric conduction voltage value as the non-input detection number increases. The present modifications can improve the degree of freedom in the configuration.

Space cleaning device 1000 in the present exemplary embodiment includes electrolysis tank 200 and humidification tank 300. However, the present disclosure is not limited to this, and for example, electrolysis tank 200 and the humidification tank 300 may be integrated as a water storage. The present modification can simplify the structure of space cleaning device 1000.

In the present exemplary embodiment, water shortage float 260 detects a water shortage by the position of the magnet in the float. However, the present disclosure is not limited to this, and for example, a water shortage may be detected based on the number of times of supply of the electrolyzed water with fixed capacity container 224. For example, when electrolysis tank 200 is 1000 ml and fixed capacity container 224 is 250 ml, the water shortage is detected when the electrolyzed water is supplied four times with fixed capacity container 224. The present modification can increase the degree of freedom in the configuration.

In the present exemplary embodiment, water or electrolyzed water is supplied when a water shortage is detected. However, the present disclosure is not limited to this, and for example, next supply may be performed after a certain period of time from when water or electrolyzed water is supplied. The present modification can increase the degree of freedom in the configuration.

Controller 500 may further include a storage that stores control content currently in execution. An example of the storage is a nonvolatile memory. Controller 500 periodically stores, into the storage as necessary, the control content currently in execution. When the power supply is restored after the power supply is shut off in space cleaning device 1000, controller 500 may control space cleaning device 1000 based on the control content in execution stored in the storage. That is, when the power supply is restored after the power supply has been shut off in space cleaning device 1000, controller 500 resume from the control content that has been in execution stored in the storage. This makes it possible to perform correct control content even when the power supply is restored after the power supply has been shut off in space cleaning device 1000.

### REFERENCE MARKS IN THE DRAWINGS

100 water storage tank
110 water supply tank
112 lid
120 first pump
122 first water supply pipe
124 supply port
128 first supplier
130 second pump
132 second water supply pipe
138 second supplier
160 water shortage float
200 electrolysis tank
210 electrodes
220 third pump
222 third water supply pipe
224 fixed capacity container
226 third water supply pipe
228 third supplier
240 supply region
250 full water float
260 water shortage float
300 humidification tank
310 cleaner
350 full water float
360 water shortage float
370 drainage float
400 electrolysis accelerator inputter
404 input port
410 electrolysis accelerator
420 case
422 case cover
424 rotator
426 motor
428 bearing hole
430 convex surface
432 rotation shaft
434 cutout
440 hole
442 guide tube
450 light emitter
452 light receiver
460 electrolyzed water generator
500 controller
510 input instruction unit
512 input detector
514 non-input detection number counter
520 electric conduction processor
522 electric conduction setting determiner
530 notifier
1000 space cleaning device

What is claimed is:

1. A space cleaning device comprising:
an electrolysis tank that stores water and electrolyzed water;
an electrolysis accelerator inputter that inputs an electrolysis accelerator into the electrolysis tank;
an electrolyzed water generator that generates the electrolyzed water by electrolyzing the water into which the electrolysis accelerator is input;
a cleaner that brings the electrolyzed water generated by the electrolyzed water generator into contact with air sucked from an intake port;
an input instruction unit that instructs the electrolysis accelerator inputter to input the electrolysis accelerator;
an input detector that detects input of the electrolysis accelerator by the electrolysis accelerator inputter;
a non-input detection number counter that counts a non-input detection number that is a number of times the input detector has not detected input of the electrolysis accelerator even after the input instruction unit has instructed the input of the electrolysis accelerator; and
an electric conduction setting determiner that determines electric conduction setting for electrolysis in the electrolyzed water generator based on the non-input detection number counted by the non-input detection number counter.

2. The space cleaning device according to claim 1, wherein
the input instruction unit instructs the electrolysis accelerator inputter to input the electrolysis accelerator until the input detector detects the input of the electrolysis accelerator, and
the electrolyzed water generator generates the electrolyzed water by the electric conduction setting after the input detector detects the input of the electrolysis accelerator.

3. The space cleaning device according to claim 1, wherein
the electric conduction setting includes setting of an electric conduction time in the electrolyzed water generator, and
the electric conduction setting determiner shortens the electric conduction time as the non-input detection number increases.

4. The space cleaning device according to claim 1, wherein
the electric conduction setting includes setting of an electric conduction current value in the electrolyzed water generator, and
the electric conduction setting determiner decreases the electric conduction current value as the non-input detection number increases.

5. The space cleaning device according to claim 1, wherein
the electric conduction setting includes setting of an electric conduction voltage value in the electrolyzed water generator, and
the electric conduction setting determiner decreases the electric conduction voltage value as the non-input detection number increases.

6. The space cleaning device according to claim 1, wherein the input instruction unit does not instruct the input of the electrolysis accelerator by the electrolysis accelerator inputter when the non-input detection number becomes equal to or greater than a threshold.

7. The space cleaning device according to claim 6, further comprising a notifier that notifies an input error by the electrolysis accelerator inputter when the non-input detection number becomes equal to or greater than the threshold.

8. The space cleaning device according to claim 1, further comprising:
a controller that controls operation of the space cleaning device; and
a storage that stores control content currently in execution by the controller,
wherein the controller controls the space cleaning device based on the control content currently in execution stored in the storage when a power supply is restored after the power supply has been shut off in the space cleaning device.
